(19) United States
(12) Patent Application Publication
(10) Pub. No.: US 2004/0166134 A1
(43) Pub. Date: Aug. 26, 2004

(54) Animal repellent containing a combination of extract of clove oil and mustard

(76) Inventors: Roger C. Koos, Tangent, OR (US); Kevin J. McVeigh, Brownsville, OR (US)

Correspondence Address: Roger C. Koos, 30929 Seven Mile Lane, Tangent, OR, 97389, US

(21) Appl. No.: 10/371,651

(22) Filed: Feb. 24, 2003

(51) Int. Cl. A61K 35/78; A01N 65/00

(52) U.S. Cl. 424/405; 424/778; 424/755

(57) ABSTRACT

A repellent composition for repelling mice, rats, and other chewing and gnawing animals which deters nesting and habitation in agricultural seeds and agricultural products stored in bulk and in separate containers made from paper, burlap, cotton, plastic, wood and steel. The repellent composition is between 1 to 10% concentration of the natural concentration of commercial clove oil extract and commercially produced mustard. The composition can be sprayed and sprinkled on the ground, on the storage bags and containers, and it maintains effectiveness over an extended period of time.

### BACKGROUND OF THE INVENTION

Animal Repellants

Current U.S. Class: 424/760

International Class: AO1N065/00

Field of Search: 210/633

### REFERENCES CITED (REFERENCED BY)

U.S. Pat. Nos.:

6,159,474 Dec. 12, 2000

5,402,597 Apr. 4, 1995

4,668,294 May 26, 1987

4,661,504 Apr. 28, 1987

1. The invention relates to a method of repelling animals, to repellent concentrations and a method of repellent application.

2. Repellents have been used in attempts to protect stored agricultural products in containers and in bulk storage.

3. The specific repellent varies depending on the species of animal, bird or insect, which it is to repel.

4. U.S. Pat. No. 4,668,294 used thujone oil with a liquid or solid carrier such as lacquer, alcohol, sodium silicate, paraffin or sawdust to repel rodents. Thujone oil is a derivative of the cedar tree and is a natural repellent.

5. U.S. Pat. No. 5,402,597 uses extract of the herb camomile as a rodent repellent. The form of camomile used or the manner of placing the camomile in the area where rodents are not desired is not critical to the effectiveness of the camomile. The amount of camomile used is not critical. It is only necessary that a sufficient amount be used. The camomile repellent is effective for several months and loses potency with time. Reapplication may be required to maintain repellency.

6. U.S. Pat. No. 4,661,504 uses denatronium saccaride, a known bittering agent, as an animal repellent at low concentrations. It is used at 0.001 to 0.5% by weight of the composition. It is applied with a penetrating solvent and water. The bitter taste deters many species of chewing and gnawing animals.

### BRIEF SUMMARY OF THE INVENTION

1. It is the objective of this invention to provide a repellent for chewing and gnawing animals which utilizes a naturally occurring plant derivative.

2. This product repels effectively and has minimum toxicity to the animals or to the individuals making the application.

3. Clove oil and mustard are natural repellents with unique strong odors and flavors. The odor is long lasting and not detrimental to the environment.

4. The repellent is effective when applied in a combination at a concentration of 1 to 10% of the natural concentration of commercial clove oil extract and commercially produced mustard.

5. Sprays of the combination extracts of clove oil and mustard repellent in a 1 to 10% aqueous solution provided rodent repellency for 3 months in a rodent accessible commercial seed storage facility containing bagged grass seed. Mice and rats were present in the non-treated bags of seed and had chewed the untreated bags of grass seed.

6. Paper and plastic seed storage bags containing grass seed were sprayed with the repellent. These bags were undamaged by rodent activity three months later. Untreated bags were damaged by rodent chewing and feeding.

### BRIEF DESCRIPTION OF THE SEVERAL VIEWS OF THE DRAWING

‘Not Applicable’

### DETAILED DESCRIPTION OF THE INVENTION

1. Clove oil is a common plant oil and mustard is a common plant extract. Clove oil is used for a variety of topical medical purposes. Clove oil has been used in topical applications on diseased teeth to lessen pain. It is used in cooking applications in some SE Asian cultures. Mustard is used in cooking applications in many cultures.

2. Clove oil and mustard have strong smells and distinctive tastes which chewing and gnawing rodents find undesirable. When a sufficient amount is sprayed in an aqueous solution upon seed in storage bags, bulk containers, and wooden pallets, the rodents will not eat the seed or chew the seed bags. Its odor and taste repel the rodents. The amount of clove oil and mustard used in the concentrations useful in this invention are non-toxic to people and domestic animals. The clove oil and mustard repels the rodents.

3. The form of clove oil used is an aqueous solution of 1 to 10% concentration of the natural concentration of commercial clove oil. This concentration is sold commercially as “one-fold” concentration of natural clove oil. The form of mustard used is an aqueous solution of 1% to 10% concentration of the commercially produced mustard.

4. The scent and taste are the critical components. The most efficient method to protect seed in bags has been to spray the outside of the bags with the aqueous solution. Wooden pallets containing the treated seed bags also are sprayed with the combination of clove oil and mustard.

5. The amount of clove oil and mustard is not critical. It is only necessary that a sufficient amount be used. Clove oil and mustard are the active ingredients of the repellent.

6. In seed storage buildings treated with clove oil and mustard combination, rodents have left for other dwelling sites. The repellency has been effective for up to 3 months. Repeated applications will maintain the effectiveness of this repellent over time.

It will be understood that the above embodiments are for purposes of illustration only. Changes and modifications may be made without departure from the spirit and scope of the invention.

We claim:

1. A repellent composition for animals comprising the placing of an effective amount of extract of clove oil and commercially produced mustard in an area where rodents are not desired and on storage containers made of paper, burlap, cotton, other fiber products, plastic, wood and steel.

2. The repellent is effective at the concentration of 1 to 10% of the natural concentration of commercial clove oil extract and commercially produced mustard.

3. The repellent can be applied in a 1 to 10% aqueous solution form when it is sprayed at 1 to 10% concentration of the natural concentration of commercial clove oil and commercially produced mustard.

4. The repellent is environmentally safe and has an effectiveness lasting up to 3 months after application.
